SEQUENCE LISTING (1 sequence)

SEQ ID NO: 1 - protein, 164 aa, Homo Sapien
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

(19) United States
(12) Patent Application Publication
(10) Pub. No.: US 2020/0171051 A1
(43) Pub. Date: Jun. 4, 2020

(54) METHODS FOR TREATING RENAL INJURY BY THERAPEUTICALLY UPREGULATING P21

(71) Applicant: RENIBUS THERAPEUTICS, INC., Lantana, TX (US)

(72) Inventors: Richard A. ZAGER, Mercer Island, WA (US); Donald Jeffrey KEYSER, Southlake, TX (US); Alvaro F. GUILLEM, Lantana, TX (US)

(21) Appl. No.: 16/697,808

(22) Filed: Nov. 27, 2019

Related U.S. Application Data
(60) Provisional application No. 62/773,743, filed on Nov. 30, 2018.

(51) Int. Cl. A61K 31/573; A61K 47/54; A61K 47/64

(52) U.S. Cl. CPC A61K 31/573; A61K 47/64; A61K 47/542

(57) ABSTRACT

The present invention relates to methods for treating acute kidney injury that utilize a glucocorticoid prodrug. The glucocorticoid prodrug selectively delivers a glucocorticoid to the kidney, where it is capable of eliciting a p21 protective response. The present invention also contemplates several novel glucocorticoid prodrugs capable of use in the above manner.

### RELATED APPLICATIONS

The present application claims benefit from U.S. Provisional Patent Application Ser. No. 62/773,743 filed Nov. 30, 2018, the contents of which are hereby incorporated by reference.

### INCORPORATION BY REFERENCE A SEQUENCE LISTING

The contents of the Sequence Listing named ST25.txt which was created on Feb. 10, 2020 and is 1,751 bytes in size is hereby incorporated by reference in its entirety.

### BACKGROUND OF THE INVENTION

The kidney is responsible for water and solute excretion from the body. Its functions include maintenance of acid-base balance, regulation of electrolyte concentrations, control of blood volume, and regulation of blood pressure. As such, loss of kidney function through injury and/or disease results in substantial morbidity and mortality. A detailed discussion of renal injuries is provided in Harrison's Principles of Internal Medicine, 17.sup.th Ed., McGraw Hill, New York, pages 1741-1830, which are hereby incorporated by reference in their entirety. Renal disease and/or injury may be acute or chronic. Acute and chronic kidney disease are described as follows (from Current Medical Diagnosis & Treatment 2008, 47.sup.th Ed, McGraw Hill, New York, pages 785-815, which are hereby incorporated by reference in their entirety): “Acute renal failure is worsening of renal function over hours to days, resulting in the retention of nitrogenous wastes (such as urea nitrogen) and creatinine in the blood. Retention of these substances is called azotemia. Chronic renal failure (chronic kidney disease) results from an abnormal loss of renal function over months to years”.

Acute renal failure (ARF, also known as acute kidney injury, or AKI) is an abrupt (typically detected within about 48 hours to 1 week) reduction in glomerular filtration. This loss of filtration capacity results in retention of nitrogenous (urea and creatinine) and non-nitrogenous waste products that are normally excreted by the kidney, a reduction in urine output, or both. It is reported that ARF complicates about 5% of hospital admissions, 15-30% of cardiopulmonary bypass surgeries, and up to 35% of intensive care admissions. ARF may be categorized as prerenal, intrinsic renal, or post-renal in causation. Intrinsic renal disease can be further divided into glomerular, tubular, interstitial, and vascular abnormalities.

Also known as cyclin-dependent kinase inhibitor 1, or CDK-interacting protein 1, p21 is a cyclin-dependent kinase inhibitor (CKI) that is capable of inhibiting all cyclin/CDK complexes. The amino acid sequences for human p21 is as follows:

(SEQ ID NO. 1)
MSEPAGDVRQNPCGSKACRRLFGPVDSEQLSRDCDALMAGCIQEA
RERWNFDFVTETPLEGDFAWERVRGLGLPKLYLPTGPRRGRDELG
GGRRPGTSPALLQGTAEEDHVDLSLSCTLVPRSGEQAEGSPGGPGD
SQGRKRRQTSMTDFYHSKRRLIFSKRKP

P21 represents a major target of p53 activity and thus is associated with linking DNA damage to cell cycle arrest. AKI-induced renal p21 elevations can exert diverse renal cytoprotective effects. Nath K A. Provenance of the protective property of p21. Am J Physiol 2005; 289: F512-513. This has been most convincingly demonstrated by studies showing that p21 deficient mice have increased sensitivity to both toxic and ischemic renal damage. Megyesi J, Andrade L, Vieira J M Jr, Safirstein R L, Price P M. Coordination of the cell cycle is an important determinant of the syndrome of acute renal failure. Am J Physiol 2002; 283: F810-F816; Price P M, Safirstein R L, Megyesi J. The cell cycle and acute kidney injury. Kidney Int. 2009; 76:604-613; Yu F, Megyesi J, Safirstein R L, Price P M. Identification of the functional domain of p21WAF1/CIP1 that protects cells from cisplatin cytotoxicity. Am J Physiol 2005; 89: F514-F520.

There is a need to find ways to pharmacologically up-regulate renal tubular p21 in order to provide therapeutic kidney protection.

### SUMMARY OF THE INVENTION

In one embodiment, the invention involves a method for treating acute kidney injury. The method includes administering to a patient in need thereof a glucocorticoid prodrug to the patient's kidney in a therapeutically effective amount for treatment of kidney injury, the glucocorticoid prodrug comprising a glucocorticoid, a linker, and a delivery moiety. The glucocorticoid may be delivered selectively to the patient's kidney.

In one aspect, the glucocorticoid is hydrocortisone, cortisone, prednisone, prednisolone, methylprednisolone, dexamethasone, betamethasone, triamcinolone, and fludrocortisone acetate. In one preferred embodiment the glucocorticoid is dexamethasone.

In another aspect the linker that links the glucocorticoid to the delivery moiety may be an aminobutyrate linker covalently coupled to the glucocorticoid through a biodegradable ester linkage.

In another aspect, the delivery moiety is a protein that may be selectively absorbed in the kidney. The delivery moiety may be lysozyme, cystatin-C, NGAL, α-1-microglobulin, or HO-1.

In one aspect, the invention involves a glucocorticoid prodrug where the prodrug comprises a glucocorticoid, a linker, and a protein that is selectively absorbed in the kidney. The prodrug may be administered by injection. The glucocorticoid may be hydrocortisone, cortisone, prednisone, prednisolone, methylprednisolone, dexamethasone, betamethasone, triamcinolone, and fludrocortisone acetate. In one preferred aspect, the glucocorticoid is dexamethasone. In one embodiment the linker is an aminobutyrate linker covalently coupled to the glucocorticoid through a biodegradable ester linkage. In one aspect, the protein of the prodrug may be lysozyme, cystatin-C, NGAL, or HO-1.

In one aspect, the invention involves a dexamethasone prodrug comprising a dexamethasone covalently linked to a protein that is selectively absorbed in the kidney. The dexamethasone may be linked to the protein through an aminobutyrate linker covalently coupled to the dexamethasone through a biodegradable ester linkage.

### BRIEF DESCRIPTION OF THE DRAWINGS

FIG. 1A shows plasma p21, urine p21, and cortical p21 after glycerol injection.

FIG. 1B shows correlation between plasma and renal cortical p21.

FIG. 1C shows correlation between renal cortical p21 mRNA values correlated with the degree of glycerol induce AKI.

FIG. 2A compares glycerol-induced increases in renal cortical p21 production 4 and 18 hours after glycerol injection.

FIG. 2B demonstrates no correlation between p21 mRNA and p53 mRNA.

FIG. 3A compares the effect of cortisol injection and dexamethasone injection 4 hours after glycerol injection.

FIG. 3B shows mifepristone (MFP) normalized p21 levels after glycerol injection returning them to the levels observed in control mice.

FIG. 4 shows lack of a significant difference in the degree of p21 protein increases in the right contralateral kidneys vs the left post ischemic (I/R) kidneys.

FIG. 5 shows one method for covalently linking dexamethasone to a protein.

### DETAILED DESCRIPTION OF THE INVENTION

Heme iron driven oxidative stress is the dominant mechanism underlying the glycerol model of rhabdomyolysis ARF. Nath K A, Balla G, Vercellotti G M, Balla J, Jacob H S, Levitt M D, Rosenberg M E. Induction of heme oxygenase is a rapid, protective response in rhabdomyolysis in the rat. J Clin Invest. 1992 90:267-270; Zager R A: Rhabdomyolysis and myohemoglobinuric acute renal failure. Kidney Int. 1996 February; 49(2):314-26.

The present study indicates for the first time that a concomitant of this oxidant injury is a dramatic increase in p21 protein levels, whether assessed in plasma, urine, or renal cortex. It is noteworthy that these changes were associated with ˜4 fold increases in renal cortical p21 mRNA, a change that mirrored AKI severity (assessed by BUN/Cr levels). Thus, increased renal cortical p21 gene transcription and translation were almost certainly at play. However, we have previously observed that the systemic effects of AKI can increase p21 gene activity in extra-renal organs (e.g. heart, brain). Johnson A C, Zager R A. Plasma and urinary p21: potential biomarkers of AKI and renal aging. Am J Physiol 2018; Aug. 1. doi: 10.1152/ajprenal.00328.2018. Given that circulating p21 undergoes glomerular filtration, both renal as well as extra-renal p21 production likely contributed to the observed increases in renal cortical and urinary p21 concentrations. Id.

P53 is a dominant transcription factor that drives p21 gene expression (e.g. ref. 8). Given that oxidant stress is a critical p53 gene activator Liu D, Xu. p53, oxidative stress, and aging. Antioxid Redox Signal. 2011; 15(6):1669-78, it was surprising that p53 mRNA levels modestly decreased, rather than increased, at a time (4 hrs) of glycerol-induced increases in renal cortical p21 expression. Further dissociating p53 from p21 gene induction were findings that the p53 inhibitor, pifithrin-α, had no impact on glycerol-induced renal p21 mRNA elevations.

Given that multiple inflammation-induced transcription factors (e.g., NF-κB) and cytokines (e.g. IL-6) can activate p21 without p53, and given that AKI induces both renal and extra-renal inflammation, we next questioned whether the potent anti-inflammatory agent dexamethasone would suppress glycerol-mediated renal p21 gene induction. Basile J R, Eichten A, Zacny V, Münger K. NF-kappaB-mediated induction of p21(Cip1/Waf1) by tumor necrosis factor alpha induces growth arrest and cytoprotection in normal human keratinocytes. Mol Cancer Res. 2003; 4: 262-270; Bellido T, O'Brien C A, Roberson P K, Manolagas S C: Transcriptional Activation of the p21WAF1,CIP1,SDI1 Gene by Interleukin-6 Type Cytokines. J Biol Chem 1998; 273:21137-21144.

Surprisingly, the exact opposite was the result: dexamethasone dramatically increased, rather than decreased, renal cortical p21 mRNA and protein levels, both in normal and glycerol-injured kidneys. Cortisol administration fully reproduced the latter response, indicating that a glucocorticoid class effect, rather than a dexamethasone-specific action, was at play. To the best of our knowledge, the present results are the first to demonstrate that glucocorticoids can activate the renal p21 gene. Since p53 mRNA levels were unchanged by corticosteroid injection and glucocorticoids are known to suppress p53 mediated gene activation, our findings suggest a p53 independent mechanism. Sengupta S, Vonesch J-L, Waltzinger C, Zheng H, Wasylyk B: EMBO J 19: 6051-6064, 2000.

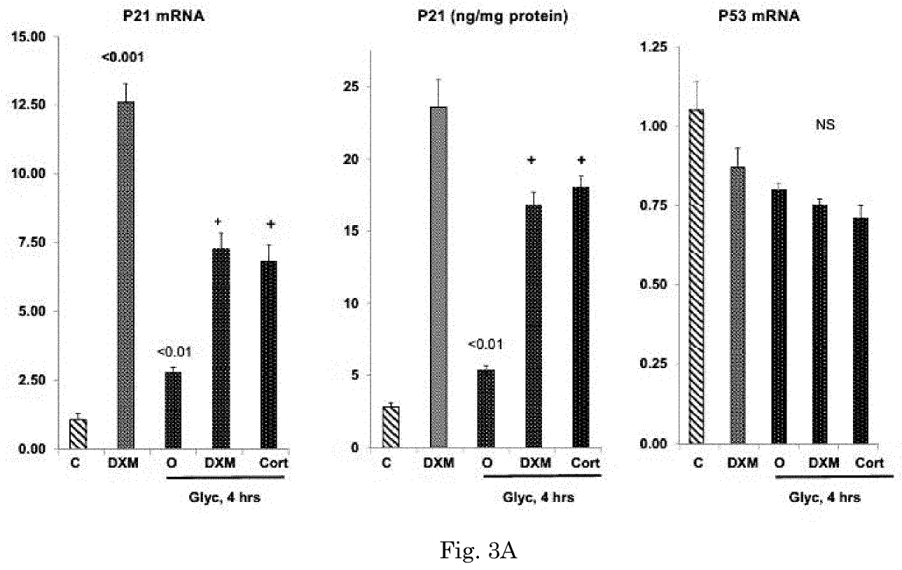

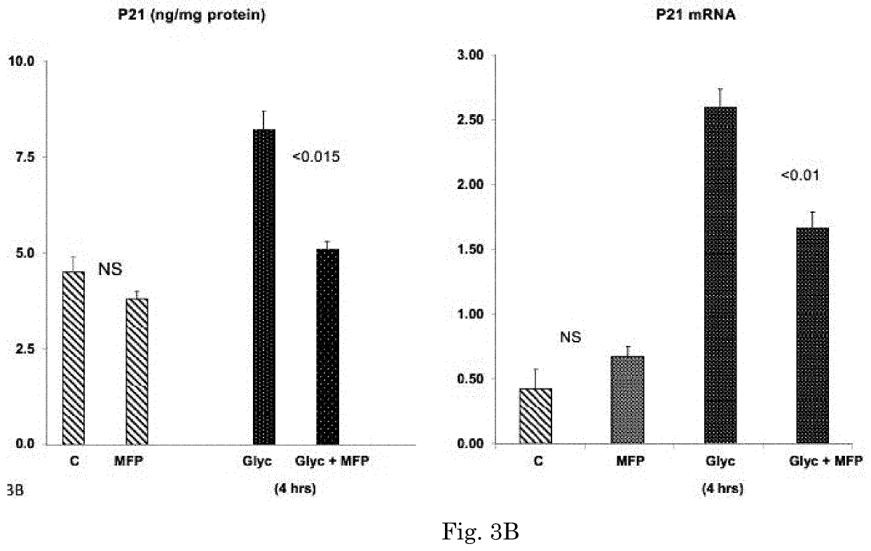

To assess whether endogenous glucocorticoid increases might help drive the glycerol-induced renal p21 increases, it was critical to determine whether the glycerol AKI model does, in fact, increase glucocorticoid production. To this end, both total plasma cortisol and urinary free cortisol levels were assessed. Within 4 hrs of glycerol injection, a 5 fold increase in total plasma cortisol was observed. Direct renal cortisol access was indicated by a 75 fold increase in urinary free cortisol at both 4 and 18 hrs post glycerol injection. To assess the physiologic relevance of these AKI-induced cortisol increases to increased p21 expression, the impact of the glucocorticoid receptor (GCR) antagonist, mefipristone, was assessed. As shown in FIG. 3B, mefipristone administration almost completely blocked glycerol-induced renal p21 increases, supporting the concept that endogenous-generated cortisol stimulates p21 gene expression and via the glucocorticoid receptor (GCR) signaling pathway. In sum, the above results suggest a novel mechanism for glycerol AKI-induced renal p21 accumulation: i) AKI induces a systemic ‘stress response’; ii) increased glucocorticoid synthesis results; iii) circulating cortisol gains ready renal access (i.e., increased urinary free cortisol levels); and iv) via interaction with the GCR pathway, increased renal p21 gene activation and p21 protein accumulation results.

Finally, we assessed whether an AKI-induced ‘stress response’ can activate the renal p21 gene even in the absence of direct tissue injury. Indeed, this appears to be the case, based on observations that mice subjected to unilateral ischemic injury demonstrated comparable p21 protein and mRNA increases in post ischemic- and uninjured (contralateral) kidneys. Furthermore, surgical stress, as induced by sham renal ischemia, also raised, albeit to a lesser degree, renal cortical p21 mRNA and protein levels. Thus, these findings indicate that systemic stress, independent of direct renal injury, is sufficient to stimulate the renal p21 pathway. The relative contribution(s) of corticosteroid- vs. potential non corticosteroid mediated ‘stress reactants’ to renal p21 gene induction remains to be defined.

In conclusion, we believe that this is the first report to document dramatic increases in both plasma and filtered glucocorticoids early in the course of experimental (glycerol-induced) AKI, and that these corticosteroid increases have the capacity to activate the renal p21 gene presumably via a p53 independent, GCR dependent, mechanism. Thus, these findings provide further insights into mechanisms of AKI induced renal p21 accumulation. Given that glucocorticoid therapy remains a mainstay in the treatment of multiple forms of both acute and chronic renal injury, the present observations suggest a novel pathway by which corticosteroids may impact these diseases. Hence, further study of this glucocorticoid-p21 pathway seems warranted, from both a basic science, as well as a clinical, perspective.

To achieve this goal, a better understanding of those factors that drive renal p21 expression during AKI are required. The present study was undertaken to help achieve this aim, using the well characterized glycerol model of oxidant-induced AKI. Nath K A, Balla G, Vercellotti G M, Balla J, Jacob H S, Levitt M D, Rosenberg M E. Induction of heme oxygenase is a rapid, protective response in rhabdomyolysis in the rat. J Clin Invest. 1992 90:267-270; Zager R A: Rhabdomyolysis and myohemoglobinuric acute renal failure. Kidney Int. 1996 February; 49(2):314-26. The information gathered offer two novel insights into AKI-mediated p21 induction: first, that AKI-induced renal p21 accumulation does not require the presence of direct renal injury; and second, that AKI induces a systemic ‘stress response’ which results in increased systemic glucocorticoid production, and that these glucocorticoid increases can activate the renal p21 gene.

The present inventors conducted studies to better understand the factors that drive renal p21 expression during AKI. All experiments were performed using male CD-1 mice (35-40 grams, Charles River Laboratories, Wilmington, Del.) maintained under routine vivarium conditions with free food and water access. The AKI models were approved by the institution's Animal Care and Utilization Committee). All surgical procedures were conducted under deep pentobarbital anesthesia (40-50 mg/Kg IP).

### EXAMPLE 1

### Glycerol AKI Model

Mice were briefly anesthetized with isoflurane, and then subjected to intramuscular glycerol injection (administered in equally divided doses into each hind limb). The glycerol dose ware varied (6, 6.5, 7, 7.5, 8, 8.5, 9 ml/Kg; n˜2-3 per dose) in order to produce variable degrees of renal injury. At 4 hrs post glycerol injection, a tail vein tail vein blood sample (˜25 μl) was obtained, and at 18 hrs, the mice were deeply anesthetized with pentobarbital. The abdominal cavity was opened through a midline abdominal incision, a terminal vena cava blood sample was obtained, the kidneys were removed, iced, and renal cortical samples were cut and extracted for both protein and total RNA (RNeasy Mini+; Qiagen; Germantown, Md.). A terminal urine sample was collected from the urinary bladder. Plasma samples were assayed for blood urea nitrogen (BUN) and creatinine (Cr). Johnson A C, Zager R A. Plasma and urinary p21: potential biomarkers of AKI and renal aging. Am J Physiol 2018; Aug. 1. doi: 10.1152/ajprenal.00328.2018. [Epub ahead of print]. Renal cortical, plasma, and urinary p21 concentrations were determined with a ‘sandwich’ ELISA which employs two distinct monoclonal antibodies (capture; detection) directed against two different p21 epitopes, conferring p21 specificity; (#2120721; Abcam, Cambridge, Mass.). Renal cortical p21 mRNA levels were determined by RT-PCR, factored by GAPDH. Ten normal mice provided control plasma, urine, and tissue values for comparisons.

To assess renal cortical changes at an earlier time point than that noted above (18 hrs post glycerol injection), an additional 5 mice were subjected to glycerol injection (8.5 ml/Kg) and 4 hrs later, renal cortical protein/RNA samples were obtained for use in the above noted assays. The results were contrasted to those observed in 5 normal mice.

### EXAMPLE 2

### Pifithrin p53 Inhibition

Pifithrin-α is a stable, water soluble, p53 inhibitor which blocks activation of p53 responsive genes. Dagher P C, Mai E M, Hato T, Lee S Y, Anderson M D Karozos S C Mang H E, Knipe N L, Plotkin Z, Sutton T A: The p53 inhibitor pifithrin can stimulate fibrosis in a rat model of ischemic acute kidney injury. Am J Physiol Renal Physiol 302: F284-F291, 2012; Komarov P G, Komarova E A, Kondratov R V, Christov-Tselkov K, Coon J S, Chernov M V et al. A chemical inhibitor of p53 that protects mice from the side effects of cancer therapy. Science 285: 1733-1737, 1999. As such, it has been used to differentiate p53-dependent vs. p53-independent pathways. To further explore the potential role of p53 on p21 gene activation, 3 mice were injected IP with 10 mg/Kg pifithrin-α (10% DMSO in saline; EMD Millipore #506154; Burlington, Mass.). Three additional mice received vehicle injection. Immediately thereafter the mice were injected with 8.5 mg/Kg glycerol. Four hrs post glycerol injection, renal cortical tissues were analyzed for p21 and p53 mRNAs.

### EXAMPLE 3

### Impact of Dexamethasone (DXM) on Glycerol-Induced p21 Elevations

Inflammation can induce p21 gene activation via the inflammatory cascade (1, 2). Basile J R, Eichten A, Zacny V, Münger K. NF-kappaB-mediated induction of p21(Cip1/Waf1) by tumor necrosis factor alpha induces growth arrest and cytoprotection in normal human keratinocytes. Mol Cancer Res. 2003; 4: 262-270; Bellido T, O'Brien C A, Roberson P K, Manolagas S C: Transcriptional Activation of the p21WAF1,CIP1,SDI1 Gene by Interleukin-6 Type Cytokines. J Biol Chem 1998; 273:21137-21144. Given that AKI evokes both intrarenal and systemic inflammatory responses, the potential for the potent anti-inflammatory corticosteroid, DXM, to alter glycerol-induced p21 gene activation was assessed. Ten mice were injected with 8.5 ml/Kg glycerol, half with and without dexamethasone injection (250 μg IP in saline; Sigma #D1159; St Louis, Mo.; administered 30 min before glycerol injection). Four hrs post-glycerol, renal cortical tissues were obtained and assayed for p21 mRNA, p53 mRNA, and p21 protein levels, as above.

To determine the impact of DXM on renal p21 expression in the absence of renal injury, 5 mice were injected with DXM as above, and 4 hrs later p21 protein/mRNA levels were measured and compared to normal values.

### EXAMPLE 4

### Cortisol (CORT) Treatment

To ascertain whether a second glucocorticoid could recapitulate DXM's p21 effects in the glycerol AKI model, 5 mice were injected with a biologically equivalent dose of cortisol (1 mg/Kg IP) followed 30 min later by 8.5 ml/Kg glycerol injection. Four hrs later, p21 protein and mRNA levels were assessed and compared to both normal values and values in 4 kidneys obtained 4 hrs post glycerol injection.

### EXAMPLE 5

### Endogenous Plasma and Urinary Cortisol Levels

To determine whether the AKI-induced systemic stress response is associated with hyper-adrenalism, urinary free cortisol levels were measured in samples from normal mice, and from mice at either 4 or 18 hrs post glycerol injection (n, 5 each; ELISA; Enzo #AD1-900-071; Farmingham, N.Y.). Johnson A C, Zager R A. Plasma and urinary p21: potential biomarkers of AKI and renal aging. Am J Physiol 2018; Aug. 1. doi: 10.1152/ajprenal.00328.2018. [Epub ahead of print]. Total plasma cortisol levels (˜95% protein bound/˜5% free cortisol) were also measured at 4 hrs post glycerol injection and compared to normal plasma values (n, 4 mice each; Clinical Chemistry Laboratories; University of Washington, Seattle, Wash.).

### EXAMPLE 6

### Impact of the Glucocorticoid Receptor Antagonist, Mifepristone (MFP), on Glycerol-Induced Renal p21 Accumulation

Given an unexpected finding that corticosteroid injection increased, rather than suppressed, p21 expression, and given that endogenous cortisol levels were markedly elevated post-glycerol injection, we questioned whether glucocorticoid receptor (GCR) blockade would decrease p21 expression. To this end, 10 mice were injected with 8.5 mg/Kg glycerol, half with and half without the GCR antagonist mifepristone (30 mg/Kg; in 85% propylene glycol; Fisher Scientific #AC459982500; ref. 10, 15). Four hrs later, the kidneys were removed and renal cortices were analyzed for p21 mRNA and protein levels. Four mice, treated with mifepristone in the absence of glycerol injection, served to evaluate its effects in the absence of glycerol injection.

### EXAMPLE 7

### Unilateral Renal Ischemia Model

Eight mice were anesthetized with pentobarbital and subjected to a midline abdominal incision, exposing the renal pedicles. Half of the mice were subjected to left renal pedicle occlusion at 37 oC×22 min by application of an atraumatic microvascular clamp. The remaining 4 mice served as surgical controls. Following completion of unilateral ischemia, the vascular clamps were removed, the abdominal cavities were closed, and the mice were allowed to recover from anesthesia. Four hrs later, they were re-anesthetized and both kidneys were resected. Kidney samples from 4 normal (non surgical subjected) mice provided control tissue samples. P21 protein and mRNA levels were compared between: i) the post-ischemic left kidneys; ii) the contralateral right kidneys; iii) kidneys from sham operated mice: and iv) from normal (non surgical) mice were assessed.

Statistics: All results are given as means±1 SEM. Statistical comparisons were made by unpaired Student's t test. If multiple comparisons were made the Bonferroni correction was applied. Significance was judged by a p value of <0.05.

### EXAMPLE 8

### Glycerol-Induced Changes in p21 Expression

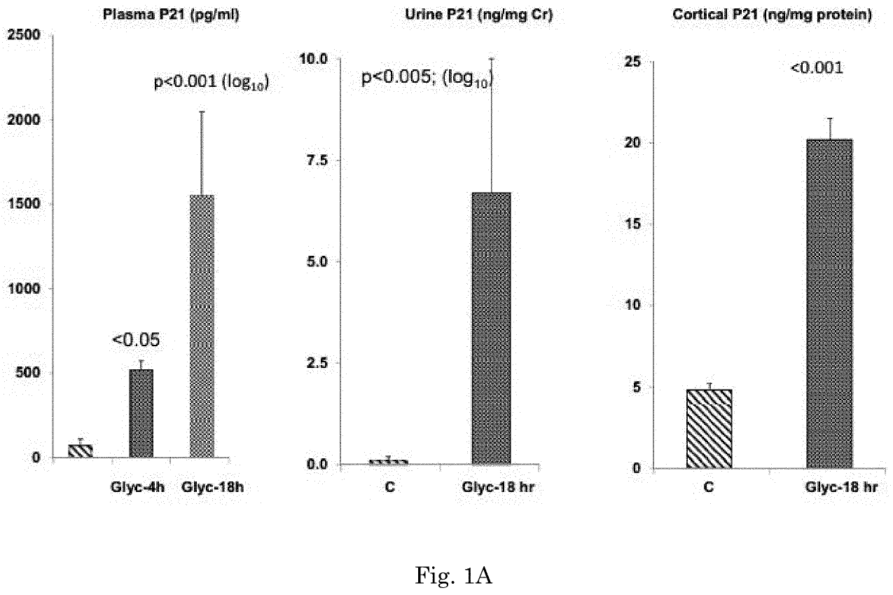

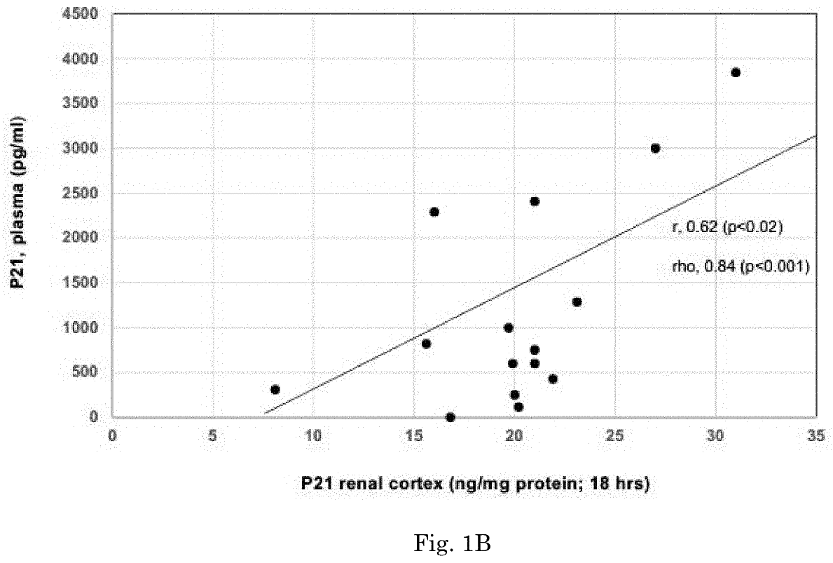

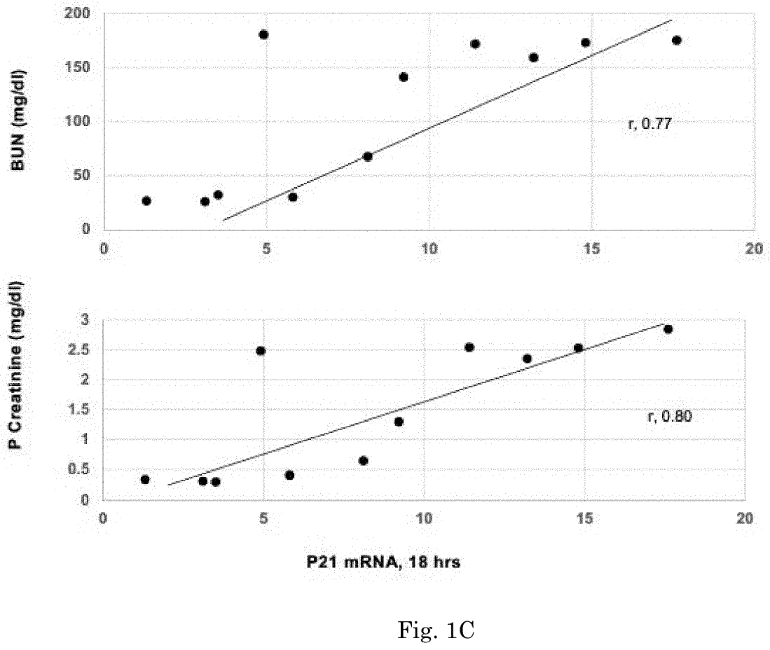

Within 4 hrs of glycerol injection, an approximate 10 fold increase in plasma 21 levels was observed (FIG. 1A, left panel).

This change was progressive in nature, with plasma p21 levels reaching values that were ˜150 fold greater than those seen in control plasma samples. The 18 hr plasma increases were matched by ˜100 fold increases in urinary p21 levels, factored by urine creatinine (FIG. 1A, middle panel).

Renal cortical p21 protein elevations were also observed at 18 hrs post glycerol injection (FIG. 1A, right panel), although the increases were relatively modest in degree (˜4× vs. controls) compared to 18 hr plasma and urinary p21 protein levels. Nevertheless, statistically significant correlations between plasma and renal cortical p21 concentrations were observed (FIG. 1B).

Renal cortical p21 mRNA values also correlated with the degree of glycerol-induced AKI, as induced by variable doses of glycerol injection (p21 mRNA vs. BUN, r, 0.77; vs plasma creatinine, r, 0.80; see FIG. 1C).

### EXAMPLE 9

### Glycerol-Induced Changes in p21 and p53 mRNA Expression

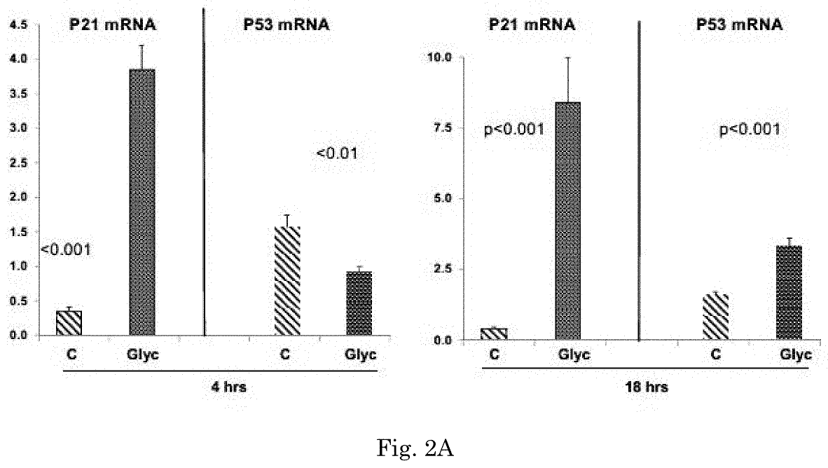

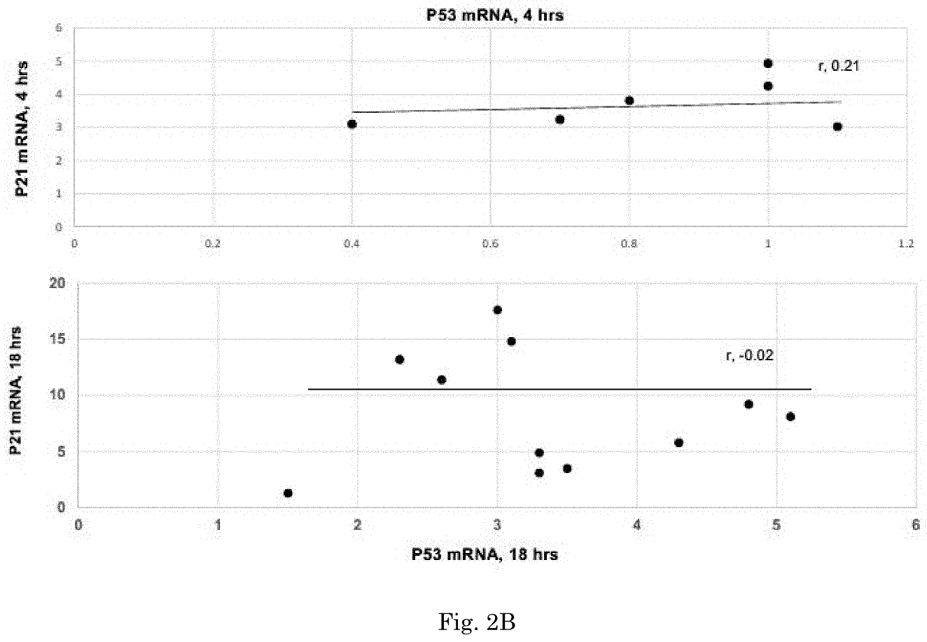

Glycerol-induced increases in renal cortical p21 production were implied by marked and progressive increases in renal cortical p21 mRNA (˜10× and ˜20× at 4 and 18 hrs post glycerol injection vs. controls; FIG. 2A; note change in y axis).

These increases could not be ascribed to p53 gene activation, given that p53 mRNA levels were decreased, rather than increased, at 4 hrs post glycerol injection (p<0.01). At 18 hrs post glycerol, slight p53 mRNA elevations were observed (FIG. 2A, right). Notably however, no correlations were between p21 mRNA and p53 mRNA were observed (r values from −0.02 to +0.2; FIG. 2B).

### EXAMPLE 10

### Pifithrin

The p53 inhibitor, pifithrin-α failed to suppress the glycerol-induced p21 mRNA increases, implying a p53 independent mechanism for p21 gene activation was at play (p21 mRNA: controls, 0.34±0.1; 4 hr glycerol, 1.43±0.11; 4 hr glycerol+pifithrin, 1.71±0.08). Pifithrin also did not impact post glycerol p53 mRNA (glycerol, 0.75±0.02 vs. glycerol+pifithrin, 0.7±0.03; NS).

### EXAMPLE 11

### Dexamethasone (DXM) Treatment

Within 4 hrs of DXM injection, dramatic increases in renal cortical p21 protein (10×) and p21 mRNA (15×) levels were observed in normal mice (FIG. 3A).

DXM also evoked ˜3 fold increases in p21 mRNA and p21 protein in 4 hr post glycerol kidneys. (FIG. 3A). In neither the normal or the 4 hr post glycerol mice could these DXM-induced p21 mRNA/protein increases be ascribed to p53 gene induction, given that p53 mRNA levels were slightly decreased with DXM treatment (FIG. 3A, right panel). Cortisol injection fully recapitulated dexamethasone's effect, raising 4 hr post glycerol p21 mRNA and p21 protein levels to the same degree as did DXM injection (FIG. 3A).

### EXAMPLE 12

### Cortisol Measurements

Within 4 hrs of glycerol injection, plasma total cortisol levels rose ˜5 fold over control values (0.9±0.1 vs 0.16±0.1 μg/ml; p<0.0001). These changes were accompanied by massive (˜75 fold) increases in urinary free cortisol concentrations at both 4 and 18 hrs post glycerol injection (normal urine, 3.1±0.5 ng/ml; 4 hrs post glycerol, 205±131 ng/ml; 18 hrs post-glycerol, 180±152 ng/ml; both time points p<0.01 vs normal values). When factored by urine creatinine concentrations, dramatic post-glycerol urine cortisol increases were still observed (log base 10 value conversions: controls, 0.7±0.1; 4 hrs 1.7±0.4; p=0.01; 18 hrs, 1.6±0.4; p=0.01 vs controls).

### EXAMPLE 13

### Glucocorticoid Receptor Antagonist, Mifepristone (MFP), Effects

By 4 hrs post MFP administration, no significant decrease in p21 protein or mRNA values were observed in control mice (FIG. 3B).

In contrast, in glycerol treated mice, MFP normalized p21 protein levels in glycerol mice, returning them to values in control kidneys. A modest reduction in post glycerol p21 mRNA levels was also observed (FIG. 3B).

### EXAMPLE 14

### P21 Expression in Sham Operated Mice and Unilateral Renal Ischemia Mice

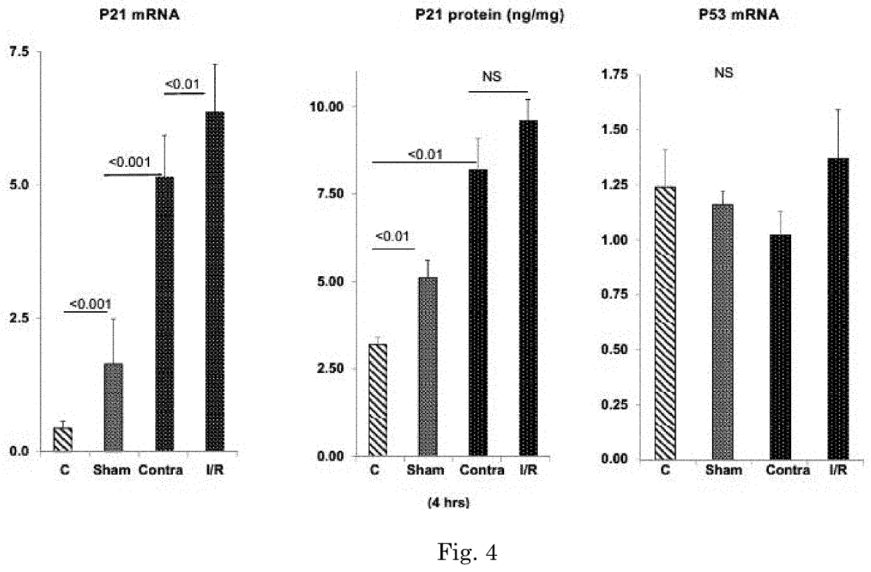

As shown in FIG. 4 left panel, within 4 hrs of performing sham surgery a 4 fold increase in renal cortical p21 mRNA was observed.

By 4 hrs post unilateral (I/R) ischemic renal injury, both the post ischemic and contralateral kidneys had marked p21 mRNA elevations (10× and 15× respectively). These p21 mRNA increases corresponded with marked increases in p21 protein levels. Of note was the lack of a significant difference in the degree of p21 protein increases in the right contralateral kidneys vs the left post ischemic (I/R) kidneys (FIG. 4). Renal p53 mRNA remained unchanged in these sham surgery or unilateral ischemia experiments.

The following examples illustrate various embodiments of the invention utilizing different delivery methods of dexamethasone. Dexamethasone cannot be systemically administered due to side effects outside of the kidney. However, the present inventors that dexamethasone is capable of providing a p21 protective effect in the kidney. Therefore, the invention can be implemented by placing dexamethasone in a form that allows non-systemic administration of dexamethasone to the kidney. This typically involves forming a prodrug of dexamethasone and a protein carrier.

Other glucocorticoids besides dexamethasone may be used to achieve the same objectives, and the delivery vehicle allows for safe delivery of high glucocorticoid potency compounds with higher half-life to the kidney than would be otherwise available. Other glucocorticoids that may be used with the invention include hydrocortisone, cortisone, prednisone, prednisolone, methylprednisolone, betamethasone, triamcinolone, and fludrocortisone acetate.

| | Glucocorticoid | Terminal half-life |
|---|---|---|
| Name | potency | (hours) |
| Cortisol (hydrocortisone) | 1 | 8 |
| Cortisone | 0.8 | 8 |
| Prednisone | 3.5-5 | 16-36 |
| Prednisolone | 4 | 16-36 |
| Methylprednisolone | 5-7.5 | 18-40 |
| Dexamethasone | 25-80 | 36-54 |
| Betamethasone | 25-30 | 36-54 |
| Triamcinolone | 5 | 12-36 |
| Fludrocortisone acetate | 15 | 24 |

### EXAMPLE 15

### Administration of Dexamethasone-Lysozyme Prodrug

Dexamethasone is administered to a patient in a form that increases its delivery to a patient's kidney while reducing the amount that is delivered systemically throughout the patient's body. Dexamethasone is covalently linked to lysozyme to form a dexamethasone-lysozyme prodrug. Lysozyme has been demonstrated to deliver covalently linked drugs (captopril) to the kidney so that the drug does not have adverse systemic effects. Windt W A, Prakash J, Kok R J, Moolenaar F, Kluppel C A, de Zeeuw D, van Dokkum R P, Henning R H. Renal targeting of captopril using captopril-lysozyme conjugate enhances its antiproteinuric effect in adriamycin-induced nephrosis. J Renin Angiotensin Aldosterone Syst. 2004 December; 5(4):197-202.

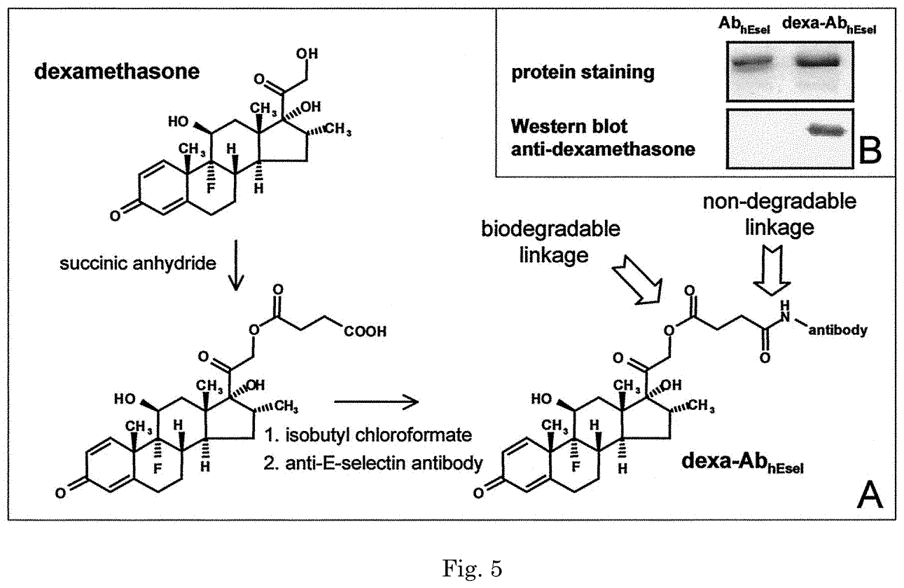

Methods of linking dexamethasone to proteins are known in the art. See Everts et al., “Selective Intracellular Delivery of Dexamethasone into Activated Endothelial Cells Using an E-Selectin-Directed Immunoconjugate,” J. Immunol, 168 (2) pp. 883-889 <available at https://doi.org/10.4049/jimmunol.168.2.883>. Everts teaching of linking dexamethasone to protein compounds is incorporated by reference in its entirety. FIG. 5 reproduced herein shows one way in which dexamethasone can be linked to a protein through a biodegradable linkage. A person having ordinary skill would have found this process of linking dexamethasone to any protein carrier (aside from an antibody as taught by Everts) to be routine chemistry in light of Everts.

This illustrates a linker that includes a link to the protein (antibody) via an amide bond that is non-degradable, and a biodegradable link to the dexamethasone through an ester bond. This forms a prodrug comprising dexamethasone, linker, and protein. Given the similarity in structure of glucocorticoids, the same chemistry can be used to prepare a linker. The linker of the present invention may include a butyramide covalently linked through a biodegradable ester linkage to the dexamethasone as shown above. The linkage may be butyl- as show above, but could alternatively be ethyl-, propyl-, or pentyl- or higher order hydrocarbon. Alternatively, the linkage may include a PEG moiety of various length in order to modulate biodegradation of the prodrug in the kidney.

In this embodiment, a patient suffering from acute kidney injury is administered dexamethasone-NGAL conjugate (i.e., prodrug) in an effective amount. The administration can be injectable, for example intramuscular or subcutaneous. The prodrug may be self-administered by the patient once a week.

### EXAMPLE 16

### Administration of Dexamethasone-Cystatin C

Dexamethasone is administered to a patient in a form that increases its delivery to a patient's kidney while reducing the amount that is delivered systemically throughout the patient's body. Dexamethasone is covalently linked to Cystatin C in this embodiment. Cystatin C may be used to deliver covalently linked drugs to the kidney similar to lysozyme so that the drug does not have adverse systemic effects.

In this embodiment, a patient suffering from acute kidney injury is administered dexamethasone-Cystatin C conjugate in an effective amount. The administration can be injectable, for example intramuscular or subcutaneous. The conjugate may be self-administered by the patient once a week.

### EXAMPLE 17

### Administration of Dexamethasone-NGAL

Dexamethasone is administered to a patient in a form that increases its delivery to a patient's kidney while reducing the amount that is delivered systemically throughout the patient's body. Dexamethasone is covalently linked to neutrophil gelatinase-associated lipocalin (NGAL) in this embodiment. NGAL be used to deliver covalently linked drugs to the kidney so that the drug does not have adverse systemic effects.

In this embodiment, a patient suffering from acute kidney injury is administered dexamethasone-NGAL conjugate in an effective amount. The administration can be injectable, for example intramuscular or subcutaneous. The conjugate may be self-administered by the patient once a week.

### EXAMPLE 18

### Administration of Dexamethasone-Alpha-1-Microglobulin

Dexamethasone is administered to a patient in a form that increases its delivery to a patient's kidney while reducing the amount that is delivered systemically throughout the patient's body. Dexamethasone is covalently linked to α-1-microglobulin in this embodiment. Alpha-1-microglobulin may be used to deliver covalently linked drugs to the kidney so that the drug does not have adverse systemic effects.

In this embodiment, a patient suffering from acute kidney injury is administered dexamethasone-α-1-microglobulin conjugate in an effective amount. The administration can be injectable, for example intramuscular or subcutaneous. The conjugate may be self-administered by the patient once a week.

### EXAMPLE 19

### Administration of Dexamethasone-HO-1

Dexamethasone is administered to a patient in a form that increases its delivery to a patient's kidney while reducing the amount that is delivered systemically throughout the patient's body. Dexamethasone is covalently linked to hemi-oxygenase (HO-1) in this embodiment. HO-1 may be used to deliver covalently linked drugs to the kidney so that the drug does not have adverse systemic effects. In this embodiment, a patient suffering from acute kidney injury is administered dexamethasone-HO-1 conjugate in an effective amount. The administration can be injectable, for example intramuscular or subcutaneous. The conjugate may be self-administered by the patient once a week.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims

What is claimed is:

1. A method for treating acute kidney injury comprising administering to a patient in need thereof a glucocorticoid prodrug to the patient's kidney in a therapeutically effective amount for treatment of kidney injury, the glucocorticoid prodrug comprising a glucocorticoid, a linker, and a delivery moiety.

2. The method of claim 1, wherein upon administration the glucocorticoid is delivered selectively to the patient's kidney.

3. The method of claim 1, wherein the glucocorticoid is hydrocortisone, cortisone, prednisone, prednisolone, methylprednisolone, dexamethasone, betamethasone, triamcinolone, and fludrocortisone acetate.

4. The method of claim 1, wherein the glucocorticoid is dexamethasone.

5. The method of claim 1, wherein the linker is an aminobutyrate linker covalently coupled to the glucocorticoid through a biodegradable ester linkage.

6. The method of claim 1, wherein the delivery moiety is a protein that is selectively absorbed in the kidney.

7. The method of claim 1, wherein the delivery moiety is lysozyme, cystatin-C, NGAL, α-1-microglobulin, or HO-1.

8. The method of claim 1, wherein the delivery moiety is lysozyme.

9. The method of claim 1, wherein the drug is administered by injection.

10. A glucocorticoid prodrug comprising a glucocorticoid, a linker, and a protein that is selectively absorbed in the kidney.

11. The glucocorticoid prodrug of claim 10, wherein the glucocorticoid is hydrocortisone, cortisone, prednisone, prednisolone, methylprednisolone, dexamethasone, betamethasone, triamcinolone, and fludrocortisone acetate.

12. The glucocorticoid prodrug of claim 10, wherein the glucocorticoid is dexamethasone.

13. The glucocorticoid prodrug of claim 10, wherein the linker is an aminobutyrate linker covalently coupled to the glucocorticoid through a biodegradable ester linkage.

14. The glucocorticoid prodrug of claim 10, wherein the protein is lysozyme, cystatin-C, NGAL, or HO-1.

15. A dexamethasone prodrug comprising a dexamethasone covalently linked to a protein that is selectively absorbed in the kidney.

16. The dexamethasone prodrug of claim 10 wherein the dexamethasone is linked to the protein through an aminobutyrate linker covalently coupled to the dexamethasone through a biodegradable ester linkage.
